(19) United States
(12) Patent Application Publication
(10) Pub. No.: US 2022/0143072 A1
(43) Pub. Date: May 12, 2022

(54) Chitosan nitrate for use as a nitric oxide donor (NO donor)

(71) Applicant: NO-ceuticals B.V., Arnhem (NL)

(72) Inventor: Mathias Hubertus Josephus Maria Rietjens, Arnhem (NL)

(73) Assignee: NO-ceuticals B.V., Arnhem (NL)

(21) Appl. No.: 17/441,704

(22) PCT Filed: Mar. 20, 2020
(86) PCT No.: PCT/NL2020/050191

§ 371 Date: Sep. 22, 2021

(30) Foreign Application Priority Data
Mar. 22, 2019 (NL) 2022788

(51) Int. Cl. A61K 31/722; A61K 8/73; A61Q 19/08; A61P 39/06; A61P 15/10; A61P 37/08; A61P 27/02; A61P 37/06; A61P 17/00

(52) U.S. Cl. CPC A61K 31/722; A61K 8/736; A61Q 19/08; A61P 39/06; A61P 17/00; A61P 37/08; A61P 27/02; A61P 37/06; A61P 15/10

(57) ABSTRACT

The present invention relates to chitosan nitrate for use as nitric oxide donor, in particular for use in the prevention or treatment of a condition related to NO deficiency in mammals, preferably humans. Suitable conditions to be treated with chitosan nitrate include disorders related to the skin, conditions related to sexual dysfunction, conditions wherein there is a need for pain treatment, conditions being a disorder related to the eyes, nose and sinus, conditions being a disorder related to the airways, conditions related to a gastro-intestinal disorder, and conditions being a metabolic disorder. These conditions were successfully treated or reduced by the application of chitosan nitrate according to the present invention. The invention also concerns a non-therapeutic or cosmetic method for skin protection and/or for healthy aging.

### FIELD OF THE INVENTION

The present invention relates to chitosan nitrate for use as a medicament, chitosan nitrate for use as a nitric oxide donor (NO donor), specifically for use as a nitric oxide (NO)-generating agent, chitosan nitrate for use in the prevention or treatment of a condition related to NO deficiency in mammals, in particular humans. The present invention further relates to chitosan nitrate for use in the prevention or treatment of a condition which is a disorder related to the skin, wherein the condition is preferably a skin disease. The present invention also relates to chitosan nitrate for use in the prevention or treatment of a condition which is a sexual dysfunction. The present invention further relates to chitosan nitrate for use in the prevention or treatment of a condition, wherein there is a need for pain treatment. The present invention further relates to chitosan nitrate for use in the prevention or treatment of a condition which is a disorder related to the eyes, nose and sinus. The present invention further relates to chitosan nitrate for use in the prevention or treatment of a condition which is a disorder related to the airways. The present invention further relates to chitosan nitrate for use in the prevention or treatment of a condition related to a gastro-intestinal disorder. The present invention also relates to chitosan nitrate for use in the prevention or treatment of a condition which is a metabolic disorder. Additionally, the present invention relates to a cosmetic method comprising the administration of chitosan nitrate, wherein preferably chitosan nitrate is used as a NO donor. The present invention also relates to a pharmaceutical composition or cosmetic composition comprising chitosan nitrate, wherein the composition is a topical formulation, such as a cream, an emulsion, a lotion, a dermal patch, a spray, a lozenge, chewing gum, a tooth paste, a nasal spray, nasal and sinus aerosol, eye drops or an eye ointment. The pharmaceutical composition comprising chitosan nitrate may be a tablet, a capsule, a suppository, a pulmonal aerosol. The nasal and sinus aerosol or the pulmonal aerosol is preferably administered via a nebulizer.

### BACKGROUND ART

In recent years, the role of the inorganic anions nitrate and nitrite, which were previously thought to be inert end products of the endogenous nitric oxide (NO) metabolism, has become more widely appreciated. Recent studies show that nitrate and nitrite anions can also be used in vivo to form NO, by the nitrate—nitrite—nitric oxide pathway (Lundberg et al., U.S. Pat. No. 10,406,118). This pathway produces nitric oxide through the reduction of dietary nitrate, for example in the form of nitrate rich vegetables such as leafy greens, beetroot, and spinach. Humans are not capable of reducing nitrate to nitrite, which occurs exclusively by bacteria naturally present in the mouth, gut, skin and mucous membranes, i.e. the so-called nitrate-responsive bacteria.

Several studies have established that NO plays an important biological role, for example as a critical regulator of vascular homeostasis, neurotransmission, redox signalling, cell respiration, and host defence. The cardio protective effects of a diet rich in vegetables might in fact be due to NO. In view of the beneficial effects, supplementing nitrate has been extensively considered. However, supplementing nitrate poses challenges, such as providing the nitrate to the nitrate-responsive bacteria capable of nitrate to nitrite conversion. Nitrite is biologically active, nitrate is not. The nitrates in nitrate-rich vegetables are efficiently absorbed in the first part of the gastrointestinal tract. Hardly any nitrate reaches the nitrate reducing bacteria in the colon. Through the entero-salivary circulation, 25% of the blood nitrate will return to the mouth where it is partly converted to nitrite. Only after this entero-salivary recirculation, the nitrate becomes bio-activatable, and therefore can be converted into nitrite by the nitrate-responsive bacteria on the tongue. Eventually only about 5% of the originally consumed dietary nitrate will be converted into biologically active nitrite, or, after further reduction, to (derivatives of) nitric oxide.

So far, all of the known NO donors have limitations. Direct administration of NO is not practical because of its gaseous form and requires the use of a gas cylinder which may not be exposed to oxygen. Diazeniumdiolates (NONOates) are potentially toxic and form carcinogenic secondary nitrosamines with adverse biological effects resulting from the metabolic by-products, for example causing oxidative stress which results in tissue damage. S-nitrosothiols are not sufficiently stable to be used for localised and topical delivery, while the potential strengths and limitations of NO hybrid drugs are not yet known.

Both in the medical and cosmetic field, the skilled person knows and distinguishes different types or subclasses of NO donors (Liang et al., Future Sci. OA 1(1), 2015, FS054). On the one hand, there are “NO-releasing agents” which are based on a (synthetic) carrier molecule to which an active form of NO is coupled. Depending on the chosen carrier, in the presence of suitable physiological or biochemical conditions, NO is released. Examples of NO-releasing agents are N-diazenium diolates, nitroso-thiols, nitroso-hydroxyl amines, nitrosyl-metal complexes, sodium nitroprusside, NO-coupled diethyl amine, V-Pyrro/NO, spermine/NO. On the other hand, “NO-generating agents” rely on cellular metabolic pathways or the nitrate-nitrite NO pathway for generating, i.e. biosynthesising NO, rather than releasing NO from a bound form. NO-generating agents can be further distinguished into organic NO-generating agents and inorganic NO-generating agents. Both types of NO-generating agents contain a nitrite or nitrate moiety. NO-generating agents classify and are hence referred to as NO prodrugs or NO precursors.

Organic nitrates and nitrites are NO donor drugs which are currently in use to treat for example coronary artery disease. Nitroglycerin (glyceryl trinitrate), isosorbide dinitrate, isosorbide mononitrate, as well as isoamyl nitrite, isopentyl nitrite and isopropyl nitrite are examples of the class of organic NO-generating agents. NO generation occurs through certain endogenous enzymes. Extended use of nitrate vasodilators induce nitrate tolerance leading to tachyphylaxis as well as aggressive side effects including increased oxidative stress, endothelial dysfunction and cardiac autonomic dysfunction. Of the organic nitrates, nitroglycerine and its derivatives have long been used in medicine, in particular in the field of cardiology as a vasodilator. A wide range of unwanted side effects has been known, such as headache, tolerance with concomitant decrease of efficacy, oxidative stress with negative effects on the blood vessels. Nitroglycerine containing plasters or ointments are often used as an NO-generating agent, for local applications or, in the case of plasters, for systemic NO increase. Organic nitrates rely on enzymes, and generally provide a quick effect, with the downside of possible overdoses and a range of side effects. It is at present generally assumed that bacteria do not play a role in the mechanism metabolising organic nitrates.

U.S. Pat. No. 6,261,594 and US 2008/311163 disclose NO-releasing agents. EP 3120840 discloses both NO-releasing agents, i.e. S-nitroso glutathione, diazenium diolate and iron-nitrosyl complexes, and organic nitrates which form and belong to a group of NO-generating agents.

U.S. Pat. No. 6,261,594 discloses a chitosan-based polymer NO donor composition comprising a modified chitosan polymer and a nitrogen oxide [N2O2] dimer, providing for “site-specific delivery” and “controlled release” of NO under physiological conditions. The group of diazenium diolates is a bioactive form of NO which is coupled to a chitosan backbone. NO is directly released from the chitosan in an aqueous medium, depending on temperature and pH value. The chitosan-based polymer NO donor composition disclosed in U.S. Pat. No. 6,261,594 thus classifies as an NO-releasing agent.

US 2008/311163 discloses a specific cosmetic treatment method and a respective device. The disclosed cosmetic disorders are caused by chronological age, environmental factors, changes in physiological functions of skin, such as psoriasis, dermatitis, acne, cellulites, and viral and/or bacteriological attacks. The disclosed device comprises a nitric oxide (NO) eluting polymer arranged to contact the area to be cosmetically treated, such that a cosmetic dose of NO is eluted from said NO eluting polymer to the treated area. The NO eluting polymer is integrated with a carrier material, which in use regulates and controls the elution of the cosmetic dosage of NO. Suitable NO-releasing polymers are chosen from (among others) the group of amine-functionalised chitosan. Chitosan is thus a carrier coupled to an active form of NO, i.e. diazenium diolate, or an s-nitrosylated or an o-nitrosylated group. Depending on the carrier and the physiological conditions NO is released.

EP3120840 relates to a nitrogen oxide-releasing wound treatment film and a preparation method therefore. The nitrogen oxide-releasing film contained S-nitroglutathione, which is a nitrogen oxide donor to be spontaneously formed in the human body. The film is applicable to the human body, where nitrogen oxide is slowly released to inhibit a pathogen, which is the main cause of wound infection. As such, wound healing is expedited. Chitosan may be used as carrier for S-nitroglutathione, because of its wound healing properties, absorbing wound secretion and maintaining moisture around the wound surface.

There still remains a general need to provide compositions for use as a NO donor, especially for delayed NO release in different parts of the mammalian body, to provide compositions for use in the prevention or treatment of a condition related to NO deficiency in a mammal. Additionally, there remains a general need to provide non-therapeutic and/or cosmetic methods directed at skin protection and/or for healthy aging.

### SUMMARY OF THE INVENTION

The invention is based on the discovery that nitrate-responsive bacteria present on the skin and in the body, for example in the colon, the tongue and mucous membranes, are able to activate inorganic nitrate if bound to a carrier. Chitosan nitrate provides such a bio-activatable form of inorganic nitrate, whereas until now only salivary nitrate was known for that. Bio-activatable nitrate refers to a form of nitrate which can be converted by nitrate-responsive bacteria into nitrite following the nitrate-nitrite-NO pathway described by Lundberg et al. (U.S. Pat. No. 10,406,118). Chitosan lends itself for coupling biotechnologically different groups to create a new molecule. Second, by using a salt form of chitosan and nitrate, nitrate can be bound and delivered to sites of action, while at the same time the positively charged amine groups of the chitosan provides for a sufficient amount of interactions with the negatively charged coating of e.g. the skin pores. Because of the salt form providing ionic bonding between chitosan and nitrate, but no covalent bonding, nitrate becomes bio-activatable so that the nitrate can be delivered to anionic sites where nitrate-responsive bacteria are residing. These sites include mucous membranes, gut and skin pores which are negatively charged. The nitrate anion itself is also negatively charged and would under normal conditions not reach the nitrate-responsive bacteria. Chitosan has cationic amine groups, some of which will be bound to nitrate. However, the remaining cationic sites still provide for interactions with the mucous membranes and allow delivery of the nitrate to those sites. The cationic chitosan both carries and navigates the nitrate anion to the negatively charged environments of mucous membranes and skin pores where nitrate-reducing bacteria are residing. In this way, nitrate in chitosan nitrate salt becomes bio-activatable.

The present inventors have thus established chitosan nitrate as an inorganic NO-generating agent, and that chitosan nitrate is metabolised by bacteria in the colon, present in mucous membranes, and in the skin. Until now, only the entero-salivary route was known for the nitrate-nitrite-NO pathway. Chitosan nitrate, however, opens the door to a unique and alternative route, wherein the nitrate is provided in a novel saliva-independent bio-activatable (SIBA) form.

The use of chitosan nitrate allows for simple and cheap production on the one hand, and provides a medically safe and efficient agent for NO delivery on the other hand. The facultative anaerobic symbiotic bacteria present in the skin and in the mammalian body provide for a controlled and sustained generation of NO from nitrate via the nitrite intermediate. Because of the slow, continuous and long-term conversion of nitrate into NO, low and medically safe NO concentrations are provided.

The present inventors have developed a medicament based on chitosan nitrate, in particular for the prevention or treatment of a condition related to NO deficiency in mammals, preferably humans. The inventors surprisingly found that chitosan nitrate was suitable as NO donor, and more specifically as a NO-generating agent. Without being bound to a theory, it is believed that chitosan nitrate is converted into nitrite by anaerobic bacteria, present for example in the skin, mucous membranes or the gastro-intestinal tract of subjects. Nitrite is further converted into nitric oxide (NO) by various pathways as known in the art. As such, application of chitosan nitrate in those areas where anaerobic bacteria are present leads to the formation of NO, which is absorbed by the body and leads to the prevention or treatment of a condition related to NO deficiency. The chitosan nitrate according to the present invention is believed to give controlled generation and release of NO after microbial activation. For example, topical NO mainly acts at the locations where it is generated. In the gastro-intestinal tract, NO acts both locally, but can also diffuse through the intestinal wall and thereby, at least partially, reach the blood stream. This effect may be confined to a few millimetres. In case of re-oxidation to nitrite and nitrate, enabling further migration or diffusion in the human body or animal, NO may become available at even more remote locations, and thus act systemically. As evidenced in the examples, several conditions related to NO deficiency, such as disorders related to the skin, conditions related to sexual dysfunction, conditions wherein there is a need for pain treatment, conditions being a disorder related to the eyes, nose and sinus, conditions being a disorder related to the airways, conditions related to a gastro-intestinal disorder, conditions being a metabolic disorder, were successfully treated or reduced by the application of chitosan nitrate according to the present invention. Additionally, the present inventors have developed a non-therapeutic or cosmetic method comprising the administration of chitosan nitrate, specifically a method for skin protection, preferably skin protection against environmental influences, and/or for healthy aging.

Accordingly, the present invention relates to chitosan nitrate for use as a medicament. The present invention also relates to chitosan nitrate for use as an NO donor, in particular for use as an NO donating medicament. This use can also be worded as the use of chitosan nitrate as NO donor or as a method of donating NO to a subject, comprising the administration of chitosan nitrate to the subject. In the uses and methods according to the present invention, chitosan nitrate may be referred to as an active ingredient. The present invention also relates to chitosan nitrate for use in the prevention or treatment of a condition related to NO deficiency in mammals. The present invention also relates to a non-therapeutic and/or cosmetic method comprising the administration of chitosan nitrate, specifically a method for skin protection, preferably skin protection against environmental influences, and/or for healthy aging. The present invention also relates to a pharmaceutical composition comprising chitosan nitrate, wherein the composition is a topical formulation, such as a cream, an emulsion, a lotion, a dermal patch, a spray, a lozenge, chewing gum, a tooth paste, a nasal spray, nasal and sinus aerosol, eye drops or an eye ointment. Alternatively, the composition is a gastro-intestinal formulation, such as a tablet, a capsule or a suppository. Else, the composition is a pulmonal aerosol. Such formulations are ideally suited to administer the chitosan nitrate at a location in the human (body) or mammal where anaerobic bacteria are present and capable of generating NO. The nasal and sinus aerosol or the pulmonal aerosol is preferably administered via a nebulizer.

### DETAILED DESCRIPTION

The present invention centres around the finding that chitosan nitrate is an efficient NO donor. Chitosan nitrate thus acts as a nitric oxide prodrug, being metabolized via the nitrate-nitrite-NO pathway after microbial activation. The present invention is further conceived on the insight that nearly all human systems rely on NO and its indirect or direct effects for homeostasis.

### Chitosan Nitrate

Chitosan is a polymer, comprising D-glucosamine and N-acetyl-D-glucosamine monomeric residues, which are randomly distributed and β-(1→4)-linked. Chitosan nitrate is also known as CTS-HNO3. Chitosan can be obtained by treating fungal chitin obtained from mushrooms, alternatively obtained from Aspergillus niger or the chitin shells of shrimp and other crustaceans, with an alkaline substance, like sodium hydroxide. Chitosan has found a versatile range of uses, including biomedical uses. Chitosan can be used in agriculture as a seed treatment, biopesticide and fungicide. Medical applications include the use in bandages to reduce bleeding, use as an antibacterial agent, and use as a drug delivery vehicle.

Luo and Wang reviewed recent development of chitosan-based polyelectrolyte complexes with natural polysaccharides for drug delivery (Int J Biol Macromol. 64, 2014, 353-67). Chitosan as a positively charged polysaccharide has been a popular biopolymer for development of drug delivery systems for various applications, due to its promising properties, including high biocompatibility, excellent biodegradability, low toxicity, as well as abundant availability and low production cost. Delivery systems fabricated from natural biopolymer-based polyelectrolyte complexes (PEC) formed by electrostatic interactions between two oppositely charged biopolymers gained general interest. In order to tailor specific applications of chitosan-based PEC drug delivery systems, various forms have been developed, including nanoparticles, microparticles, beads, tablets, gels, as well as films and membranes. Drug delivery applications of chitosan-based PEC with other natural polysaccharides, including alginate, hyaluronic acid, pectin, carrageenan, xanthan gum, gellan gum, gum arabic, and carboxymethyl cellulose have been developed.

In chitosan nitrate, the primary amine moieties of the D-glucosamine monomers are protonated to form an ammonium cation, which have nitrate as counter ion. Chitosan nitrate is a solid and readily crystallized. Thus, in one embodiment, the chitosan nitrate is in crystal or powder form. Chitosan nitrate according to the invention consists of both nitrate anions and chitosan cations, wherein the weight ratio between nitrate anions and chitosan is between 1:1 and 1:10, more preferably between 1:1 and 1:5, yet more preferably between 1:2 and 1:5, even more preferably between 1:2 and 1:4, yet even more preferably between 1:2.5 and 1:3.5. In the most preferred embodiment, the weight ratio between nitrate anions and chitosan is about 1:3. In a preferred embodiment, at least 75 mol %, more preferably at least 90 mol %, most preferably substantially all of the primary amine moieties of the D-glucosamine monomers of the chitosan are protonated and functionalised with nitrate.

The chitosan preferably has a weight-average molecular weight in the range of 20-500 kDa, more preferably in the range of 150-250 kDa, most preferably between 190 and 210 kDa. In the most preferred embodiment, the molecular weight of chitosan is about 200 kDa. The inventors found that within these molecular weight ranges, especially around 200 kDa, the chitosan nitrate gave optimal results in terms of reduction of conditions associated with NO deficiency. Furthermore, such a polymer length gave optimal results in application to the skin, when comprised in a topical formulation. Optimal reception into the pores of the skin, where the relevant anaerobic bacteria are residing, is achieved within these molecular weight ranges, such that inorganic nitrate is microbially activated and NO is readily formed and taken up by the body.

Chitosan nitrate is typically obtained by treatment of chitosan with nitric acid. In a preferred embodiment, the chitosan nitrate is obtainable by a process comprising: (a) obtaining chitin from a natural source, such as crustaceans, fungi, such as mushrooms and/or Aspergillus niger; (b) optional treatment of the chitin with an acid, such as a HCl solution, and then treatment with a base, such as a KOH solution, to obtain an intermediate product comprising chitosan, which may be isolated if desired; (c) the (intermediate) chitosan product is then contacted with nitric acid (HNO3) to obtain chitosan nitrate. The obtained chitosan nitrate is a salt that easily crystallises from solution. The chitosan nitrate may then optionally be washed and dried.

In an alternative embodiment, the chitosan nitrate is obtainable by an enzymatic process comprising enzymatic treatment of chitin with chitinase enzyme, which enzyme is commercially available.

Alternatively, chitosan nitrate can be synthesised as described in Huo, L. et al.,

Electrorheological properties of chitosan nitrate suspension, Colloids and Surfaces A: Physicochem. Eng. Aspects 316 (2008) pages 125-130. First, 2 cm³ of nitric acid (16 mol L⁻¹) was dissolved in 20 cm³ acetone, and then 2.5 g chitosan was sufficiently mixed with the solution under stirring. After drying for 4 h at 50° C. to remove acetone, the chitosan nitrate particles were ground, and dried in a vacuum oven at 50° C. for 2 days. The chitosan nitrate material was thus obtained.

In a preferred embodiment of the above described syntheses, an excess of nitric acid is used, such that substantially all primary amine moieties of the D-glucosamine monomers of the obtained chitosan nitrate are protonated and functionalised with nitrate.

### Pharmaceutical Composition

In one embodiment, the invention concerns a pharmaceutical composition comprising chitosan nitrate. Pharmaceutical compositions comprising chitosan nitrate can be prepared according to common procedures known in the art. The pharmaceutical composition according to the present invention can take any form, preferably one of an enteral composition, such as a tablet, a capsule or a suppository, a pulmonal aerosol, and a topical formulation, such as a cream, an emulsion, a lotion, a dermal patch, a spray, a lozenge, chewing gum, a tooth paste, a nasal spray, nasal and sinus aerosol, eye drops or an eye ointment. In case the composition takes the form of an emulsion, a water-in-oil emulsion is preferred, as that resulted in optimal results on treating NO deficiency related conditions. Alternatively, an oil-in-water emulsion also provides good treatment results. The nasal and sinus aerosol or the pulmonal aerosol is preferably administered via a nebulizer.

The pharmaceutical composition according to the invention comprises the chitosan nitrate as defined above. In a preferable embodiment according to the invention, the pharmaceutical composition comprising chitosan nitrate having a molecular weight in the range of 20-500 kDa, more preferably in the range of 150-250 kDa, most preferably between 190 and 210 kDa.

The composition according to the invention preferably comprises vitamin C (ascorbic acid). If present, the vitamin C is preferably comprised in the composition in an amount of 0.5-50 wt %, more preferably 1-20 wt %, most preferably 2.5-10 wt %, based on chitosan nitrate. In an especially preferred embodiment, the composition comprises vitamin C and a phosphate salt as defined herein below.

In one embodiment of the invention, the pharmaceutical composition is a topical formulation, such as a cream. In one embodiment, a cream comprises a water-in-oil (W/O) emulsion. In another embodiment, a cream comprises a water-in-oil-in-water(W2) (W1/O/W2) double emulsion. In yet another embodiment, a cream comprises an oil-in-water (O/W) emulsion. In the W/O emulsion, the water phase comprises the chitosan nitrate, typically in combination with a pharmaceutically acceptable salt, such as NaCl and/or a phosphate containing salt. Preferred phosphate salts are tetrasodium pyrophosphate (TSPP; Na₄P₂O₇) and/or disodium phosphate (DSP; Na₂HPO₄). Most preferably, DSP is used. The oil phase preferably comprises vaseline/paraffin. The vaseline and paraffin are preferably present in a weight ratio in the range of 2:1-1:2, more preferably in about equal parts of weight. In a preferred embodiment, the oil phase further comprises lanolin. The cream may be prepared by slowly adding the water phase to the oil phase while stirring. DSP serves the stabilisation of the chitosan nitrate particles. It is preferably employed at a weight percentage of 3-10 wt %, preferably 4-7 wt %, most preferably about 5 wt % based on chitosan nitrate.

The W/O emulsion or the W1/O1W2 double emulsion comprises preferably between 50 and 70 wt % vaseline/paraffin, between 5 and 20 wt % lanoline, between 20 and 50 wt % distilled water, between 0.1 and 5 wt % chitosan nitrate, between 0.1 and 1.0 wt % NaCl and between 3 and 10 wt % DSP based on chitosan nitrate. More preferably, the W/O emulsion or the W1/O/W2 double emulsion comprises between 55 and 65 wt % vaseline/paraffin, between 8 and 15 wt % lanoline, between 25 and 35 wt % distilled water, between 0.5 and 2 wt % chitosan nitrate, between 0.2 and 0.5 wt % NaCl, and between 4 and 6 wt % DSP based on chitosan nitrate. In a most preferable embodiment, the W/O emulsion or the W1/O/W2 double emulsion comprise about 60 wt % vaseline/paraffin, about 10 wt % lanoline, about 28.1 wt % distilled water, about 1 wt % chitosan (about 200 kDa) nitrate, about 0.3 wt % NaCl, and about 5 wt % DSP based on chitosan nitrate.

In another embodiment of the invention, the pharmaceutical composition is a tablet or a capsule, or a colon-targeted capsule. The tablets or capsules comprise preferably between 50 and 2000 mg chitosan nitrate, more preferably between 100 and 1000 mg chitosan nitrate, yet more preferably between 200 and 800 mg chitosan nitrate, most preferably between 400 and 600 mg chitosan nitrate. In a preferable embodiment, the prepared tablets or capsules contain about 480 mg chitosan nitrate which consists of about 120 mg nitrate anions and about 360 mg chitosan. In a further preferable embodiment, chitosan nitrate can be stabilised by adding phosphates, preferably dihydrogen phosphate (DSP) at a maximum of 10 wt % with respect to chitosan nitrate.

In yet another embodiment of the invention, the pharmaceutical composition is a nasal spray or nasal aerosol. The nasal spray comprises sterile water, a pharmaceutically acceptable salt, such as for example NaCl, and preferably between 0.05 and 5 wt % chitosan nitrate, more preferably between 0.1 and 2 wt % chitosan nitrate, yet more preferably between 0.3 and 1 wt % chitosan nitrate. In a preferable embodiment, the nasal spray contains sterile water, about 0.5 wt % chitosan(about 200 kDa) nitrate, about 0.9 wt % NaCl. The nasal spray or nasal aerosol is preferably administered via a nebulizer.

The following embodiments according to the invention were prepared and tested on patients suffering from various medical and medical-like conditions. The composition defined here below are the most preferred pharmaceutical compositions in the context of the present invention.

Chitosan nitrate capsules were prepared containing 480 mg chitosan nitrate which consists of 120 mg NO₃₋ anions and 360 mg chitosan 200 kDa. In a further preferable embodiment, chitosan nitrate can be stabilised by adding phosphates, preferably disodium hydrogen phosphate (DSP) at a maximum of 10 wt % with respect to chitosan nitrate.

A cream containing either a water(W)-in-oil(O) (W/O) emulsion or a water(W1)-in-oil(O)-in-water(W2) (W1/O/W2) double emulsion was prepared. In the W/O emulsion, the water phase contains chitosan (200 kDa)-nitrate and NaCl and/or DSP, and the oil phase contains vaseline/paraffin in equal parts of weight and lanolin. The cream is prepared by slowly adding the water phase to the oil phase while stirring. The emulsion contains 60 wt % vaseline/paraffin, 10 wt % lanoline, 28.1 wt % distilled water, 1 wt % chitosan (200 kDa)-nitrate, 0.3 wt % NaCl, and 0.05 wt % DSP. Yet another cream based on an O/W emulsion contains 60 wt % distilled water, 2 wt % crystal sorbitol solution, 0.15 wt % sorbic acid, 0.5 wt % chitosan nitrate; 0.025 wt % DSP, 2 wt % D-panthenol, 7 wt % glycerine; about 0.05 wt % citric acid adjusted to reach a pH of around 4.5, 7.5 wt % cetomacrogol wax, 10 wt % decyl oleate; 7 wt % fractionated coconut oil; 2 wt % shea butter, and 2 wt % BTMS-25.

Nasal spray containing sterile water, 0.5 wt % chitosan(200 kDa)-nitrate, and 0.9 wt % NaCl was prepared. A further nasal spray containing sterile water, 0.25 wt % chitosan(200 kDa)-nitrate, 2.00 wt % NaCl, 0.00125 wt % Na₂HPO₄, 0.05 wt % eucalyptus oil, and 0.0125 wt % benzalkonium chloride (used as a preservative) was also prepared.

### Cosmetic Composition

In one embodiment, the invention concerns a cosmetic composition comprising chitosan nitrate. Other than not being intended for therapeutic applications, the cosmetic compositions may be similar or even identical to the pharmaceutical compositions. Thus, all preferred embodiments for the pharmaceutical composition according to the invention equally applies to the cosmetic compositions according to the invention. The cosmetic composition may also be referred to as a non-therapeutic composition. Cosmetic compositions comprising chitosan nitrate can be prepared in the same way as the above described pharmaceutical compositions. The cosmetic compositions according to the present invention can take any form, for example an enteral composition or a (nutritional) supplement. Typically, the cosmetic composition takes the form of a tablet, a capsule or a topical formulation, such as a cream, an emulsion or a lotion. In case the composition takes the form of an emulsion, a water-in-oil emulsion is preferred, as that resulted in optimal results.

However, an oil-in-water emulsion can also be used.

The non-therapeutic and/or cosmetic composition according to the invention comprises the chitosan nitrate as defined above. In a preferable embodiment according to the invention, the pharmaceutical composition comprising chitosan nitrate having a molecular weight in the range of 20-500 kDa, more preferably in the range of 150-250 kDa, most preferably between 190 and 210 kDa.

In one embodiment of the invention, the non-therapeutic and/or cosmetic composition is a topical formulation, such as a cream. In one embodiment, a cream comprises a water-in-oil (W/O) emulsion. In another embodiment, a cream comprises a water-in-oil-in-water(W2) (W1/O/W2) double emulsion. The W/O emulsion or the W1/O/W2 double emulsion can be prepared in the same way, as described above for the pharmaceutical composition. An oil-in-water (O1W) emulsion can also be used.

In another embodiment of the invention, the non-therapeutic and/or cosmetic composition is a tablet or a capsule. The tablets or capsules comprise preferably between 50 and 2000 mg chitosan nitrate, more preferably between 100 and 1000 mg chitosan nitrate, yet more preferably between 200 and 800 mg chitosan nitrate, most preferably between 400 and 600 mg chitosan nitrate. In a preferable embodiment, the prepared tablets or capsules contain about 480 mg chitosan nitrate which consists of about 120 mg nitrate anions and about 360 mg chitosan.

The non-therapeutic and/or cosmetic composition according to the invention may optionally further contain DSP, preferably in an amount of 5-10 wt % based on the total weight of the cosmetic composition.

### Chitosan Nitrate for Use as a Medicament and Chitosan Nitrate for Use as a NO Donor

The invention relates to chitosan nitrate for use as a medicament. In an embodiment related herewith, the invention relates to chitosan nitrate for use as a NO donor. In another embodiment related herewith, the invention relates to chitosan nitrate for use in the prevention or treatment of a condition related to NO deficiency. According to the invention, it is preferable that chitosan nitrate is administered as a pharmaceutical composition comprising chitosan nitrate, wherein the pharmaceutical composition is either a topical formulation, a tablet, a capsule or a nasal spray or a pulmonal aerosol. The nasal spray or the pulmonal aerosol is preferably administered via a nebulizer.

In a preferred embodiment, to maintain or achieve desirable levels of NO, chitosan nitrate capsules containing 300-600 mg, preferably about 480 mg, chitosan nitrate are administered twice a day, preferably one capsule in the morning and one capsule in the evening, or alternatively two capsules in the morning. In an alternative embodiment, to maintain or achieve higher levels of NO on a short-term basis, chitosan nitrate capsules containing 300-600 mg, preferably about 480 mg, chitosan nitrate are administered at a dose of 6 capsules per day during a maximum of three days, wherein most preferably administration is equally distributed during the day.

In another preferable embodiment, to maintain or achieve desirable levels of NO, for topical applications using for example the cream, in the initial stages prescription/administration is twice a day, and once desirable levels of NO are established administration once a day suffices.

In another preferable embodiment, to maintain or achieve desirable levels of NO, the nasal spray is administered at initially four times per day at 1 to 2 puffs in both nostrils, and once symptoms are alleviated, the nasal spray is administered once per day at 1 to 2 puffs in both nostrils.

In one embodiment, the invention relates to chitosan nitrate for use in the prevention or treatment of a condition related to NO deficiency selected from a disorder related to the skin, a sexual dysfunction, a condition wherein there is a need for pain treatment, a disorder related to the eyes, nose and sinus, a disorder related to the airways, a gastro-intestinal disorder, a metabolic disorder. Where the condition is a skin disease, the skin disease is for example psoriasis, atopic dermatitis, acne vulgaris, cold sores and/or shingles, the latter also known as zoster or herpes zoster. Alternatively, the skin condition is a vascular problem as seen in Raynaud syndrome, specifically scleroderma and/or chilblains. Further alternatively, the skin condition is resulting from oxidative stress due to UV radiation (photodamage) and/or skin aging, in particular premature skin aging. Where the condition is a sexual dysfunction, preferably there may be a need for topical application of chitosan nitrate in the genital area for the treatment of erectile dysfunction and for the treatment of female sexual disorder (FSD). Alternatively, the condition may be such that there is a need for pain treatment, wherein preferably the condition is tendinitis, lateral epicondylitis, arthritis, fissura ani, post traumatic pain and/or swelling, or wherein there is a need for neuromodulation as part of pain treatment related to diabetic neuropathy, carpal tunnel syndrome and/or migraine.

Where the condition is a disorder related to the eyes, nose and sinus, preferably the condition is an allergic conjunctivitis, dry eyes, glaucoma, allergic rhinitis, a non-allergic rhinitis and/or a sinusitis. Where the condition is a disorder related to the airways, preferably the condition is asthma, chronic obstructive pulmonary disease (COPD) and/or arterial pulmonary hypertension. Where the condition is related to a gastro-intestinal disorder, preferably the gastro-intestinal condition or disorder is leaky gut, low diversity of gut microbiota, low Akkermansia Muciniphila, dysbiosis and/or irritable bowel syndrome (IBS). Where the condition is a metabolic disorder, preferably the metabolic disorder is hypertension, diabetes mellitus type 2 and/or atherosclerosis.

Thus, in a preferable embodiment, chitosan nitrate for use in the prevention or treatment of the above conditions is administered as a pharmaceutical composition comprising chitosan nitrate, wherein the pharmaceutical composition is a topical formulation or a tablet. In a preferable embodiment, chitosan nitrate for use in the prevention or treatment of the above conditions is administered as a topical formulation, wherein the topical formulation, for example a cream, preferably comprising a water(W)-in-oil(O) (W/O) emulsion or a water(W1)-in-oil(O)-in-water(W2) (W1/O/W2) double emulsion, comprising preferably between 50 and 70 wt % vaseline/paraffin, between 5 and 20 wt % lanoline, between 20 and 50 wt % distilled water, between 0.1 and 5 wt % chitosan nitrate, between 0.1 and 1.0 wt % NaCl and between 3 and 10 wt % DSP based on chitosan. More preferably, the W/O emulsion or the W1/O/W2 double emulsion comprises between 55 and 65 wt % vaseline/paraffin, between 8 and 15 wt % lanoline, between 25 and 35 wt % distilled water, between 0.5 and 2 wt % chitosan nitrate between 0.2 and 0.5 wt % NaCl and between 4 and 7 wt %

DSP based on chitosan. In yet another preferable embodiment, a cream based on an O/W emulsion contains 60 wt % distilled water, 2 wt % crystal sorbitol solution, 0.15 wt % sorbic acid, 0.5 wt % chitosan nitrate; 0.025 wt % DSP, 2 wt % D-panthenol, 7 wt % glycerine; about 0.05 wt % citric acid adjusted to reach a pH of around 4.5, 7.5 wt % cetomacrogol wax, 10 wt % decyl oleate; 7 wt % fractionated coconut oil; 2 wt % shea butter, and 2 wt % BTMS-25.

In a further preferred embodiment, the invention relates to chitosan nitrate for use in the prevention or treatment of the above conditions, wherein chitosan nitrate is administered as a tablet or a capsule, wherein the tablets or capsules comprise preferably between 50 and 2000 mg chitosan nitrate, more preferably between 100 and 1000 mg chitosan nitrate, yet more preferably between 200 and 800 mg chitosan nitrate, most preferably between 400 and 600 mg chitosan nitrate. In a preferable embodiment, the prepared tablets or capsules contain about 480 mg chitosan nitrate which consists of about 120 mg nitrate anions and about 360 mg chitosan (about 200 kDa).

In a further preferred embodiment, the invention relates to chitosan nitrate for use in the prevention or treatment of a disorder related to the airways, wherein preferably the condition is asthma, chronic obstructive pulmonary disease (COPD) and/or arterial pulmonary hypertension. In a preferable embodiment, chitosan nitrate for use in the prevention or treatment of a disorder related to the airways is administered as a pulmonal aerosol, wherein the pulmonal aerosol comprises sterile water, a pharmaceutically acceptable salt, such as for example NaCl, and preferably between 0.05 and 5 wt % chitosan nitrate, more preferably between 0.1 and 2 wt % chitosan nitrate, yet more preferably between 0.3 and 1 wt % chitosan nitrate. In a preferable embodiment, the pulmonal aerosol contains sterile water, about 0.5 wt % chitosan (about 200 kDa) nitrate, and about 0.9 wt % NaCl. The pulmonal aerosol is preferably administered via a nebulizer.

In a further preferred embodiment, the invention relates to chitosan nitrate for use in the prevention or treatment of a condition wherein there is a need for pain treatment, wherein preferably the condition is tendinitis, lateral epicondylitis, arthritis, fissura ani, post traumatic pain and/or swelling, or alternatively wherein there is a need for neuromodulation as part of pain treatment related to diabetic neuropathy, carpal tunnel syndrome and/or migraine. In a preferable embodiment, chitosan nitrate for use in the prevention or treatment of the above conditions is administered as a tablet or a capsule, wherein the tablets or capsules comprise preferably between 50 and 2000 mg chitosan nitrate, more preferably between 100 and 1000 mg chitosan nitrate, yet more preferably between 200 and 800 mg chitosan nitrate, most preferably between 400 and 600 mg chitosan nitrate. In a preferable embodiment, the prepared tablets or capsules contain about 480 mg chitosan nitrate which consists of about 120 mg nitrate anions and about 360 mg chitosan (about 200 kDa). In a more preferable embodiment, chitosan nitrate for use in the prevention or treatment of the above conditions is administered as a topical formulation, wherein the topical formulation, for example a cream, preferably comprises a water(W)-in-oil(O) (W/O) emulsion or a water(W1)-in-oil(O)-in-water(W2) (W1/O/W2) double emulsion, comprising preferably between 50 and 70 wt % vaseline/paraffin, between 5 and 20 wt % lanoline, between 20 and 50 wt % distilled water, between 0.1 and 5 wt % chitosan nitrate, between 0.1 and 1.0 wt % NaCl and between 3 and 10 wt % DSP based on chitosan. More preferably, the W/O emulsion or the W1/O/W2 double emulsion comprises between 55 and 65 wt % vaseline/paraffin, between 8 and 15 wt % lanoline, between 25 and 35 wt % distilled water, between 0.5 and 2 wt % chitosan nitrate, between 0.2 and 0.5 wt % NaCl, and between 4 and 7 wt % DSP based on chitosan. Alternatively, an oil-in-water (O/W) emulsion can be administered. A cream based on an O/W emulsion contains 60 wt % distilled water, 2 wt % crystal sorbitol solution, 0.15 wt % sorbic acid, 0.5 wt % chitosan nitrate; 0.025 wt % DSP, 2 wt % D-panthenol, 7 wt % glycerine; about 0.05 wt % citric acid adjusted to reach a pH of around 4.5, 7.5 wt % cetomacrogol wax, 10 wt % decyl oleate; 7 wt % fractionated coconut oil; 2 wt % shea butter, and 2 wt % BTMS-25.

In a further embodiment, the invention relates to chitosan nitrate for use in the prevention or treatment of a condition which is a disorder related to the eyes, nose and sinus, wherein preferably the condition is an allergic conjunctivitis, dry eyes, glaucoma, allergic rhinitis, a non-allergic rhinitis and/or a sinusitis. In a preferable embodiment, chitosan nitrate for use in the prevention or treatment of a disorder related to the nose and sinus, is administered as a nasal spray, wherein the nasal spray comprises sterile water, a pharmaceutically acceptable salt, such as for example NaCl, and preferably between 0.05 and 5 wt % chitosan nitrate, more preferably between 0.1 and 2 wt % chitosan nitrate, yet more preferably between 0.3 and 1 wt % chitosan nitrate. In a preferable embodiment, the nasal spray contains sterile water, about 0.5 wt % chitosan (about 200 kDa) nitrate, and about 0.9 wt % NaCl. In a preferable embodiment, chitosan nitrate for use in the prevention or treatment of a disorder related to the eyes, is administered as eye drops, wherein the eye drops comprise sterile water, a pharmaceutically acceptable salt, such as for example NaCl, and preferably between 0.05 and 5 wt % chitosan nitrate, more preferably between 0.1 and 2 wt % chitosan nitrate, yet more preferably between 0.3 and 1 wt % chitosan nitrate. In a preferable embodiment, the eye drops contain sterile water, about 0.5 wt % chitosan (about 200 kDa) nitrate, and about 0.9 wt % NaCl. A nasal spray containing sterile water, 0.25 wt % chitosan(200 kDa)-nitrate, 2.00 wt % NaCl, 0.00125 wt % Na₂HPO₄, 0.05 wt % eucalyptus oil, and 0.0125 wt % benzalkonium chloride (used as a preservative) was prepared.

In a preferable embodiment, chitosan nitrate for use in the prevention or treatment of allergic rhinitis, a non-allergic rhinitis and/or a sinusitis is administered as a nasal spray, wherein the nasal spray comprises sterile water, a pharmaceutically acceptable salt, such as for example NaCl, and preferably between 0.05 and 5 wt % chitosan nitrate, more preferably between 0.1 and 2 wt % chitosan nitrate, yet more preferably between 0.3 and 1 wt % chitosan nitrate. In a preferable embodiment, the nasal spray contains sterile water, about 0.5 wt % chitosan (about 200 kDa) nitrate, and about 0.9 wt % NaCl. A nasal spray containing sterile water, 0.25 wt % chitosan(200 kDa)-nitrate, 2.00 wt % NaCl, 0.00125 wt % Na₂HPO₄, 0.05 wt % eucalyptus oil, and 0.0125 wt % benzalkonium chloride (used as a preservative) was prepared.

Non-therapeutic and/or Cosmetic Method Comprising the Administration of Chitosan Nitrate

In another embodiment, the invention relates to a cosmetic method comprising the administration of chitosan nitrate, wherein preferably chitosan nitrate is used as a NO donor. The cosmetic method may also be referred to as a non-therapeutic method. In a preferable embodiment, the cosmetic method is directed at skin protection, preferably skin protection against environmental influences, and/or for healthy aging, Furthermore, in the cosmetic method, the lack of nitric oxide is due to aging. The cosmetic method according to the invention involves the administration of the cosmetic composition according to the invention, as defined above.

In a preferable embodiment, the cosmetic method comprises the administration of a W/O emulsion or a W1/O/W2 double emulsion comprising preferably between 50 and 70 wt % vaseline/paraffin, between 5 and 20 wt % lanoline, between 20 and 50 wt % distilled water, between 0.1 and 5 wt % chitosan nitrate, between 0.1 and 1.0 wt % NaCl and between 3 and 10 wt % DSP based on chitosan. More preferably, the W/O emulsion or the W1/O/W2 double emulsion comprises between 55 and 65 wt % vaseline/paraffin, between 8 and 15 wt % lanoline, between 25 and 35 wt % distilled water, between 0.5 and 2 wt % chitosan nitrate, between 0.2 and 0.5 wt % NaCl, and between 4 and 7 wt % DSP based on chitosan. In a most preferable embodiment, the W/O emulsion or the W1/O/W2 double emulsion comprise about 60 wt % vaseline/paraffin, about 10 wt % lanoline, about 28.1 wt % distilled water, about 1 wt % chitosan (about 200 kDa) nitrate, about 0.3 wt % NaCl, and about 5 wt % DSP based on chitosan. Alternatively, an oil-in-water (O/W) emulsion can be administered. A cream based on an O/W emulsion contains 60 wt % distilled water, 2 wt % crystal sorbitol solution, 0.15 wt % sorbic acid, 0.5 wt % chitosan nitrate; 0.025 wt % DSP, 2 wt % D-panthenol, 7 wt % glycerine; about 0.05 wt % citric acid adjusted to reach a pH of around 4.5, 7.5 wt % cetomacrogol wax, 10 wt % decyl oleate; 7 wt % fractionated coconut oil; 2 wt % shea butter, and 2 wt % BTMS-25.

### Water-in-Oil Emulsion for Topical Applications

A water-in-oil emulsion is widely employed for topical application. There are three pathways in intradermal or transdermal drug delivery, i.e. the skin appendages (or adnexa) route, also known as the appendageal route, where drugs enter through skin-associated structures, such as the skin pores, hair follicles and the sweat ducts, and furthermore the transcellular and the intercellular route. Chitosan nitrate is mainly directed at the appendageal route, hence a W/O emulsion is preferable. Skin is generally hydrophobic, whereas the appendages are hydrophilic, so that the hydrophilic moieties will easily penetrate the appendages. In these appendages, bacteria are located which are able to reduce chitosan nitrate via the nitrate-nitrite-NO pathway, also referred to as microbial bio-activation of nitrate. The hydrophobic moieties have a preference for the remaining part of the skin which itself is lipophilic. It is thus accepted that the W/O emulsion allows chitosan nitrate to enter the appendages. Nevertheless, an O/W emulsion or a cream based thereon is also suitable, because the oil covers the skin except the hydrophilic pores of the appendages. The chitosan nitrate is dissolved in the water phase of the cream.

The stratum corneum, i.e. the outermost layer of the epidermis, is lipophilic so that water cannot enter the skin. The skin pores however are hydrophilic. Chitosan nitrate is hydrophilic. A W/O emulsion enables the appendageal route for chitosan nitrate take-up. In the skin pores, there are facultative anaerobic bacteria reducing nitrate to nitrite. This is generally believed to be the first step in the nitrate-nitrite-nitric oxide pathway. In the skin pores, the ducts and hair follicles, bacterial lysozymes and bacterial chitosanases break down the chitosan moieties of the chitosan nitrate.

The inventors found that oligo chitosan (10 kDa)-nitrate, oligo chitosan (10 kDa) with nitrate salt added to it, oligo chitosan (10 kDa) with nitrite added to it, can easily cause skin irritation in some people. Areas of the skin typically affected are inside the elbows, the back of the knees and the back of the neck. It is applicant's opinion that this may be due to the accumulation of nitrite and/or chitosan in between the cornea cells. Chitosan (200 kDa)-nitrate in a W/O or W/O/W emulsion in the embodiments described herein seem to circumvent these problems. Chitosan (200 kDa)-nitrate in an O/W emulsion in the embodiments described herein also circumvents these problems. It has been found that addition of DSP in an amount of 5 wt % with respect to the chitosan nitrate makes both the W/O emulsion and the O/W emulsion even more efficient. A nitrate salt by itself without chitosan in a W/O emulsion or an O/W emulsion gives only very weak improvements, if any at all. All embodiments with nitrite and without chitosan cause itchy and inflamed skin.

### EXAMPLES

### Example 1

### High Blood Pressure (Hypertension)

A male, 65 year old person used blood pressure lowering medicines before. Without medication, his blood pressure was 145 mm Hg systolic and 100 mm Hg diastolic. He has been administered with chitosan nitrate for one year, at a dosage of one capsule administered two times per day.

The chitosan nitrate capsules contain 480 mg chitosan nitrate which consist of 120 mg NO₃_{^{−}} anions and 360 mg chitosan 200 kDa (weight-average molecular weight M_{w}).

The blood pressure of the patient became normal, i.e. 130/85 systolic/diastolic within three months. He has not experienced any side effects.

Measuring the fasting value of salivary nitrate and nitrite does give a reflection of the plasma value of nitrate and nitrite. The patient took one capsule chitosan nitrate, containing 120 mg NO₃_{^{−}} anion, at 11:00 pm. The fasting value, measured the next morning at 7:30, for salivary nitrate (NO₃_{^{−}}) was >500 mg/L and for salivary nitrite (NO₂_{^{−}}) was >80 mg/L.

After taking an equivalent amount of NO₃_{^{−}} as KNO₃ salt at 11:00 pm, containing 180 mg KNO₃ salt, the fasting value in the next morning was 80 mg/L for NO₃_{^{−}} and 4.7 mg/L for NO₂_{^{−}}.

Oligo chitosan nitrate 10 kDa was also used and tested. An equivalent amount of 120 mg NO₃_{^{−}} was taken at 11:00 pm. The fasting values in the next morning at 7:30 were 144 mg/L for NO₃_{^{−}} and 14.4 mg/L for NO₂_{^{−}}. Oligo chitosan is absorbed before reaching the colon and NO₃_{^{−}} will then also be absorbed.

Quantofix test strips nitrate and nitrite from Macherey-Nagel company, Germany, were used for measurement of NO₃_{^{−}} and NO₂_{^{−}}. These test strips allow semiquantitative measurement. Reading of the test strips was performed with a Quantofix Relax reader.

In the case of KNO₃, no NO₃_{^{−}} will reach the large intestines, because KNO₃ gets immediately absorbed and is for 75% excreted in the urine. In the case of chitosan (200 kDa)-nitrate the salivary levels of NO₃_{^{−}} and NO₂_{^{−}}, measured after 8.5 hours after administration, were relatively higher as compared to the other examples. This means that nitrate reaches the colon, at least partially, and gets converted into bioactive NO forms. NO is known to stimulate the blood circulation in the intestinal wall. An increased blood circulation can in turn enhance mucus formation on the intestinal wall and thus positively modulate the microbiome and benefit the growth of bacteria like Akkermansia muciniphila. Enhanced mucus formation strengthens the gut barrier and prevents diseases because toxic substances will not be able to enter the blood stream.

Other tests were done based on the ingestion of the daily dose in the morning and by measuring the salivary nitrate and salivary nitrite 24 hours later. Two capsules of chitosan nitrate contained an equivalent amount of 240 mg NO₃_{^{−}}. Ten measurements were done for both chitosan nitrate and KNO₃ salt. For chitosan nitrate the average fasting values in the saliva were 447 mg/l NO₃_{^{−}} and 45 mg/l NO₂_{^{−}}, and for KNO₃ respectively 274 mg/l NO₃_{^{−}} and 15.1 mg/l NO₂_{^{−}}. The scientific literature suggests that saliva nitrite values are most representative for the NO status in plasma. De nitrite values in saliva, taken 24 hours after ingestion, are for chitosan nitrate thus three times higher than when the equivalent amount of the nitrate anion is administered in the form of KNO₃.

Microbiome 16s rRNA sequencing was performed at the start of the treatment and one year later. There was an increase in the number of bacterium Akkermansia muciniphila of 166%. A. muciniphila plays an important role in overweight, obesity and diabetes mellitus.

In conclusion, chitosan nitrate has the effect of sustained higher levels of nitrate and (bioactive forms of) nitrite in the blood, as compared to KNO₃ and oligo chitosan nitrate 10 kDa. Additionally, chitosan nitrate modulates the gut microbiome in a positive way.

### Example 2

### Eczema (Atopic Dermatitis)

A girl aged 5 years has been suffering for many years from eczema on her face, neck, arms and legs with extremely itchy skin. Many corticosteroid creams had been administered to manage the symptoms. Then the treatment was changed. A cream containing chitosan nitrate has been administered for one year. The cream has been applied to the skin twice a day. Since this intervention there was only a need for a corticosteroid cream containing 0.1% Triamcinolon about two times a month.

The cream contains either a water(W)-in-oil(O) (W/O) emulsion or a W1/O/W2 double emulsion. In the W/O emulsion, the water phase contains chitosan (200 kDa)-nitrate, NaCl and DSP, and the oil phase contains vaseline/paraffin in equal parts and lanolin. The cream is prepared by slowly adding the water phase to the oil phase while stirring.

The emulsion contains 60 wt % vaseline/paraffin, 10 wt % lanoline, 28.1 wt % distilled water, 1 wt % chitosan (200 kDa)-nitrate and 0.9 wt % NaCl. In case DSP is added, then the emulsion contains 0.05 wt % DSP and 28.05 wt % distilled water.

The water-in-oil (W/O) or water-in-oil-in-water (W/O/W) emulsions further containing different ingredients and emulsifiers can be prepared according to standard techniques known in the art. Alternatively, an oil-in-water emulsion (O/W) further containing different ingredients and emulsifiers can be prepared according to standard techniques known in the art. A cream based on an O/W emulsion contains 60 wt % distilled water, 2 wt % crystal sorbitol solution, 0.15 wt % sorbic acid, 0.5 wt % chitosan nitrate; 0.025 wt % DSP, 2 wt % D-panthenol, 7 wt % glycerine, about 0.05 wt % citric acid adjusted to reach a pH of around 4.5, 7.5 wt % cetomacrogol wax, 10 wt % decyl oleate; 7 wt % fractionated coconut oil; 2 wt % shea butter, and 2 wt % BTMS-25.

### Example 3

### Cold, Stiff and Painful Hands in a Scleroderma Patient

A male patient, 50 years old, familiar with scleroderma Raynaud complaints in cold weather conditions. Cold, stiff and painful hands are symptoms due to poor circulation of blood. The patient has experienced considerable improvement after applying a W/O/W emulsion with 1 wt % chitosan(200 kDa)-nitrate three times a day, applied continuously throughout the year but less often in summertime, i.e. the patient suffered from less pain and less stiffness, and the hands of the patient felt warmer, and the patient overall feels more comfortable.

### Example 4

### Diabetic Polyneuropathy

A male patient, 66 years old, has been suffering from diabetes and severe polyneuropathy of the legs for years. The patient could not sleep at night because of leg pain. After applying the W/O/W emulsion on his legs twice a day for one month, he experienced a great improvement in his condition, i.e. pain relief. Nitric oxide is believed to modulate the small nerve fibres responsible for pain generation. As polyneuropathy is a chronic condition, lifelong treatment is required.

### Example 5

### Migraine; Neuromodulation of the Trigeminal Nerve

A woman, 35 years old, has been suffering from migraine headache with aura for about six days a month. The initial treatment consisted of topical application of the W/O emulsion containing 1 wt % chitosan(200 kDa)-nitrate on the skin of the forehead and on the eyebrows. During an attack of migraine the patient applies the emulsion up to 4 times a day, in between attacks the patient applies the emulsion once a day. This treatment is believed to positively affect the ophthalmic branch of the trigeminal nerve. During subsequent treatment, a nasal spray containing 0.5 wt % chitosan(200 kDa)-nitrate and 0.9 wt % NaCl was administered additionally twice per day during the attack of migraine. This treatment is believed to positively affect the maxillary branch of the trigeminal nerve. By way of modulating two branches of the trigeminal nerve, the number of days that the patient was suffering from (migraine) headache was reduced by more than 50 %.

In another patient suffering from both tension headache and migraine headache, topical application of the same W/O emulsion on the occipital triangle, affecting the cervico-trigeminal pathway, was added to the above mentioned treatment. No side effects were reported and there was a reduction of the number of days the patient was suffering from migraine and tension headache. Specifically, the W/O emulsion has been locally applied on the so-called occipital triangle. Additionally, local application of the W/O emulsion to the forehead is continued. At a later stage during the course of treatment, an O/W emulsion with the same amount of chitosan nitrate was applied yielding the same results.

### Example 6

### Carpal Tunnel Syndrome

A female patient, 70 years old, has been suffering from carpal tunnel syndrome affecting both wrists and hands, and caused hereby poor sleep. Decompression operation of the nerves was proposed by a neurosurgeon, but not carried out. The patient started with topical application of the W/O emulsion containing 1 wt % chitosan(200 kDa)-nitrate, initially administered twice a day for one month and later on once a day for half a year. After this treatment, the patient had no complaints anymore originating from carpal tunnel syndrome.

### Example 7

### Rhinitis

Several patients, both male and female, between 25 and 65 years old, with allergic rhinitis, complaining of an itchy nose, sneezing, nasal obstruction and rhinorrhoea have been administered nasal spray containing sterile water, 0.5 wt % chitosan(200 kDa)-nitrate and 0.9 wt % NaCl. Administration was two times a day consisting of one to two spray puff in both nostrils. Good improvements were achieved, i.e. alleviation of the above symptoms of itchy nose, sneezing, nasal obstruction and rhinorrhoea. Administration was allowed more often, if the patient felt a need for it.

Also patients with non-allergic rhinitis have been administered the nasal spray and experienced an improvement with the above defined treatment, such as less snoring and a better sleep. Decongestion of the nose and modulation of the nasal branches of the trigeminal nerve are believed to be part of the working mechanism. In allergic rhinitis mast cell stabilization may also play a role. In both allergic and non-allergic rhinitis, reduction of oxidative stress by nitric oxide is believed to play an important role in reducing the complaints resulting from rhinitis. Because of its different working mechanism in contrast to the common nasal sprays for rhinitis, the chitosan nitrate containing spray may be taken together with other sprays, if necessary.

At a later stage during treatment of both allergic and non-allergic rhinitis patients, the following composition of nasal spray was applied twice per day with good results. The nasal spray contained sterile water, 0.25 wt % chitosan(200 kDa)-nitrate, 2.00 wt % NaCl, 0.00125 wt % Na₂HPO₄, 0.05 wt % eucalyptus oil, and 0.0125 wt % benzalkonium chloride (used as a preservative) was prepared.

### Example 8

### Fissura Ani

A male patient, 56 years old, suffering from pain caused by a fissura ani, already received a prescription from his family doctor for painful anus. The prescribed ointment was isosorbide dinitrate cream 1%. The patient stopped using this cream due to serious side effects, such as severe headache and flushing. Isosorbide dinitrate is an organic nitrate like nitroglycerin which is totally different from the inorganic nitrate as in chitosan nitrate. Both types of nitrates are prodrugs for nitric oxide, but the inorganic nitrates are understood to work only locally without causing headache or flushing. The patient switched to the W/O emulsion containing 1 wt % chitosan(200 kDa)-nitrate three times a day. No side effects were reported during this treatment and healing of the fissura took place within a few weeks, after which time the treatment can be finished.

### Example 9

### Sun Radiation Protection; Promotion of Skin Tanning

Topical application of a W/O/W emulsion with 1 wt % chitosan (200 kDa)-nitrate causes a bit of sunless tanning through the nitric oxide production and fills up the nitrite stores in the skin. UV radiation converts the nitrite into nitric oxide. The W/O/W emulsion is not to be considered a sunscreen, but in applying this W/O/W emulsion two times per day three to four days before sun exposure and immediately after sun exposure as well, less damage of the skin cells through sun radiation was observed.

### Example 10

### Tennis Elbow (Lateral Epicondylitis)

A male patient, 42 years old, has been suffering from a painful right underarm and elbow for 4 months, which was diagnosed as a tennis elbow. He received physiotherapy and got one injection of 1 ml Kenacort A10 combined with 1 ml xylocaine in the tendon insertion on the lateral epicondyle. The patient started with topical application of the W/O emulsion of 1 wt % chitosan(200 kDa)-nitrate at an initial dose of two times a day. The patient experienced less pain after two weeks of application and continued applying the emulsion once a day for one month.

Example 11

### Erectile Dysfunction

A male patient, 57 years old, has been suffering from obesity, cardio-vascular problems and diabetes type 2, and erectile dysfunction for some years. Erectile dysfunction is such that erection is insufficient for penetration. Sildenafil (Viagra) tablets were contra-indicated because of the organic nitrates, such as nitro-glycerin, the patient has been using. Inorganic nitrates like chitosan nitrate are therefore allowed because they lack an interaction with PDE-5 as opposed to the PDE-5 inhibitor Sildenafil. The patient started applying the W/O/W emulsion containing 1 wt % chitosan nitrate and 0.05% DSP on the glans and penile shaft, half an hour before sexual activity. This treatment resulted in satisfying sexual intercourse in 80% of the attempts.

### Example 12

### Allergic Conjunctivitis

A female patient, 45 years old, has been suffering from hay fever, for which she has been using 5 mg Desloratadine, one tablet a day, and Levocabastine eye drops applied 3 times a day, one drop in each eye. Addition of chitosan nitrate eye drops, containing 0.5 wt % chitosan, 0.9 wt % NaCl in sterile water, applied two times per day, one drop in each eye, resulted in a strong alleviation of the symptoms of hay fever.

### Example 13

### Exercise-Induced Asthma

A male patient, 25 years old, has been suffering from house dust mite allergy. For one year he has been suffering from shortness of breath when starting with running. This symptom lasts five minutes only and then stops, which was diagnosed as exercise-induced asthma. The patient started using a Pariboy SX nebulizer. A solution of sterile water with 0.5 wt % chitosan nitrate and 0.9 wt % of NaCl was prepared. About 10 minutes before starting with running the patient inhaled 1 ml of the above-mentioned solution through the Pariboy SX nebulizer. This treatment prevented the exercise-induced asthma.

### Example 14

### A Cold Sore (Herpes Simplex Virus Infection)

A female patient, 65 year old, has been suffering from recurrent cold sore, affecting the lips and the area under the nose. Exposure to sun radiation and common colds usually caused an exacerbation of the cold sore. The patient felt an upcoming eruption of the cold sore by a sudden start of tingling in her upper lip. The patient started applying an O/W cream immediately after experiencing the described symptoms, the O/W cream having the following composition: water 60 wt %; crystal sorbitol solution 2 wt %; sorbic acid 0.15 wt %; chitosan nitrate (CTS-HNO3) 0.5 wt %; DSP 0.025 wt %; D-panthenol 2 wt %; glycerine 7 wt %; citric acid 0.05 wt % (adjusted to reach a pH of around 4.5); cetomacrogol wax 7.5 wt %; decyl oleate 10 wt %; fractionated coconut oil 7 wt %; shea butter 2 wt %; BTMS-25 2 wt %. During the first three days, the O/W cream was applied six times per day. It was observed that the cream prevented the cold sore from blistering, with the result that no crusts were formed and that the patient experienced hardly any pain. After three days of treatment, the cream has been applied less often and treatment has been stopped after another three days. The Herpes Simplex virus relies on oxidative stress for multiplication. It is believed that the strong antioxidant nitric oxide, generated from chitosan nitrate, is responsible for the achieved treatment result.

1-36. (canceled)

37. A method for treating a subject in need thereof, comprising administering to the subject a composition comprising chitosan nitrate.

38. The method according to claim 38, wherein chitosan nitrate acts as a NO donor.

39. The method according to claim 38, wherein the method is for the prevention or treatment of a condition related to NO deficiency in a mammal.

40. The method according to claim 39, wherein the mammal is a human.

41. The method according to claim 39, wherein the condition is a disorder related to the skin, a sexual dysfunction, a disorder related to the eyes, nose and sinus, a disorder related to the airways, a gastro-intestinal disorder or a metabolic disorder.

42. The method according to claim 41, wherein the condition is psoriasis, atopic dermatitis and/or acne vulgaris, a vascular problem as seen in Raynaud syndrome, scleroderma and/or chilblains; a disorder resulting from oxidative stress due to UV radiation and/or skin aging, a disorder resulting from oxidative stress due to cold sores, a sexual dysfunction wherein there is a need for topical application of chitosan nitrate in the genital area for the treatment of erectile dysfunction and for the treatment of female sexual disorder (FSD), allergic conjunctivitis, dry eyes, glaucoma, allergic rhinitis, a non-allergic rhinitis and/or a sinusitis, asthma, chronic obstructive pulmonary disease (COPD) and/or arterial pulmonary hypertension, leaky gut, low diversity of gut microbiota, low Akkermansia Muciniphila, dysbiosis and/or irritable bowel syndrome (IBS), hypertension, diabetes mellitus type 2, obesity and/or atherosclerosis.

43. The method according to claim 37, wherein the mammal is in need of pain treatment.

44. The method according to claim 43, wherein the condition is tendinitis, lateral epicondylitis, arthritis, fissura ani, post traumatic pain and/or swelling; or wherein there is a need for neuromodulation as part of pain treatment related to diabetic neuropathy, carpal tunnel syndrome and/or migraine.

45. The method according to claim 37, wherein chitosan has a molecular weight in the range of 20-500 kDa.

46. The method according to claim 45, wherein chitosan has a molecular weight in the range of 150-250 kDa.

47. A method for skin protection and/or for promoting healthy aging, comprising the administration of chitosan nitrate to a subject.

48. The method according to claim 47, wherein chitosan nitrate acts as a NO donor.

49. The method according to claim 47, wherein the skin protection is selected from protection against environmental influences and reduced skin aging.

50. The method according to claim 47, wherein the subject suffers from a lack of nitric oxide due to aging.

51. A pharmaceutical or cosmetic composition comprising chitosan nitrate.

52. The composition according to claim 51, wherein chitosan has a molecular weight in the range of 20-500 kDa.

53. The composition according to claim 52, wherein chitosan has a molecular weight in the range of 150-250 kDa.

54. The composition according to claim 51, wherein the composition is a topical formulation, a gastro-intestinal formulation or a pulmonal aerosol.

55. The composition according to claim 54, wherein the composition is a cream, an emulsion, a lotion, a dermal patch, a spray, a lozenge, chewing gum, a tooth paste, a nasal spray, nasal and sinus aerosol, eye drops, an eye ointment, a tablet, a capsule or a suppository.
